(19) United States
(12) Plant Patent Application Publication
(10) Pub. No.: US 2018/0332755 P1
(43) Pub. Date: Nov. 15, 2018

(54) Wuliang sanqi

(50) ;

(71) Applicants: Wei-He Wang, Yuxi (CN); Gou-shun Wu, Yuxi (CN); Simon Pu, La Habra Heights, CA (US); Shun-Meng Zhang, Yuxi (CN); Ming-Iun Yang, Yuxi (CN)

(72) Inventors: Wei-He Wang, Yuxi (CN); Gou-shun Wu, Yuxi (CN); Simon Pu, La Habra Heights, CA (US); Shun-Meng Zhang, Yuxi (CN); Ming-Iun Yang, Yuxi (CN)

(73) Assignee: Wuliang Herb Valley Group, Diamond Bar, CA (US)

(21) Appl. No.: 15/731,250

(22) Filed: May 9, 2017

(51) Int. Cl. A01H 6/14

(52) U.S. Cl. PLT/263.1

(57) ABSTRACT

The disclosure relates to a new and distinct Wuliang Sanqi (Latin name: Panax notoginseng (Burkill) F. H. Chen ex C. Chow & W. G. Huang) which is planted under forest.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND

The invention relates to herbs, and more particularly to a new and distinct Wuliang Sanqi (Latin name: Panax notoginseng (Burkill) F. H. Chen ex C. Chow & W. G. Huang) which is planted under forest.

Sanqi is a kind of herb which is an important drug resource belonging to ginseng species. SANQI is a perennial herbaceous plant, which is native to the southwest mountainous areas in China and is an undergrowth herbaceous plant growing under subtropical moist evergreen broad-leaved forest, wherein, according to the biological characteristics and ecological habits of Sanqi, Sanqi is very sensitive to environmental factors, including light, humidity, temperature, and soil. Wild Sanqi herb is difficult to find because of long-term excavation. Wild ginseng plants are facing the danger of endangered and extinct and the protection and sustainable use of germplasm resources of Panax species has become an urgent problem to be solved.

### SUMMARY

Provided herein is a new and distinct Wuliang Sanqi (Latin name: Panax notoginseng (Burkill) F. H. Chen) which is a cultivated geography provenance.

The Wuliang Sanqi is characterized by having a well-developed main root, fleshy hypertrophy, commonly known as “head” or “root”, one to several thick side roots on the main root, also known as “branch roots” or “ribs”, fibrous roots commonly known as “hair roots”, rhizome also known as “notch”, “lut” or “sheep intestine head”, wherein the base portion of the rhizome has thinner stem section stripes, commonly known as “parrot mouth”.

In spring of the second year, new buds sprout and the old stem of last year falls off that forms a dent on the rhizome, known as “stem marks”. Rhizome is elongated year by year, the age of the Wuliang Sanqi is known according to the number of stem marks on the rhizome.

The Wuliang Sanqi is generally has a single stem, having an enlarged stem base, commonly known as “chicken leg”, wherein the stems is generally in green color, or may also be in purple or green purple color and etc. A three-year-old Wuliang Sanqi has a plant height of 30-80 cm and a flower stalk length of 10-30 cm. The Wuliang Sanqi generally has five palmately compound leaves with each petiole having a length of 5-7 cm, and five to seven small leaves with each petiole having a length of 2 cm. The Wuliang Sanqi has leaf membranous, the central one being the largest, each having a long oval shape to an obovate-long oval shape, a length of 6.5-11 cm, a width of 2-4 cm, an acuminate to long acuminate apex, a round to wide cuneate base, leaves edge with serrates, and hairs on veins on both sides.

The Wuliang Sanqi flowers and fruits from its second year, flowering for 7 months to 9 months, wherein each of the flower stems has a length of 9-27 cm and, sometimes, a flower stem makes up about a ½ total height of the plant. Its umbel is born at the cauline top. Generally, the Wuliang Sanqi has more than one hundred of flowers, which are small, yellow-green in color, and bisexual. Each of the flowers has five calyxes, which are blade lobed and slightly triangle in shape, each having teeth at sepals margin thereof. Each of the flowers has five petals, white in color, oval shaped, and arranged in compound tile form. Each of the flowers has five stamens, stylus twice to three cleft, base grow separatedly or together, ovary at lower portion, and 2-3 loculed.

The Wuliang Sanqi fruits from September to November. The fruit is drupaceous berry, in kidney or spherical kidney shape, and has a diameter of about 6-13 mm. The matured fruit is bright red, occasionally yellow or black, shiny. Seeds 1-3 tablets.

The natural lifespan of the seeds of the Wuliang Sanqi is about 15 days. The seeds need to go through a dormant period, and then accelerating germination. It takes more than 3 years from sowing to harvest, including one year of seedling (commonly known as “sub-strip”). After the sub-strip is transplanted for 2 years, harvest is available.

### BRIEF DESCRIPTION OF THE DRAWINGS

FIG. 1 illustrates the grown plant of the variety.

FIG. 2 illustrates the planting base of the Wuliang Sanqi.

FIG. 3 illustrates the root portion under ground of the Wuliang Sanqi.

FIG. 4 illustrates the red seeds of the Wuliang Sanqi.

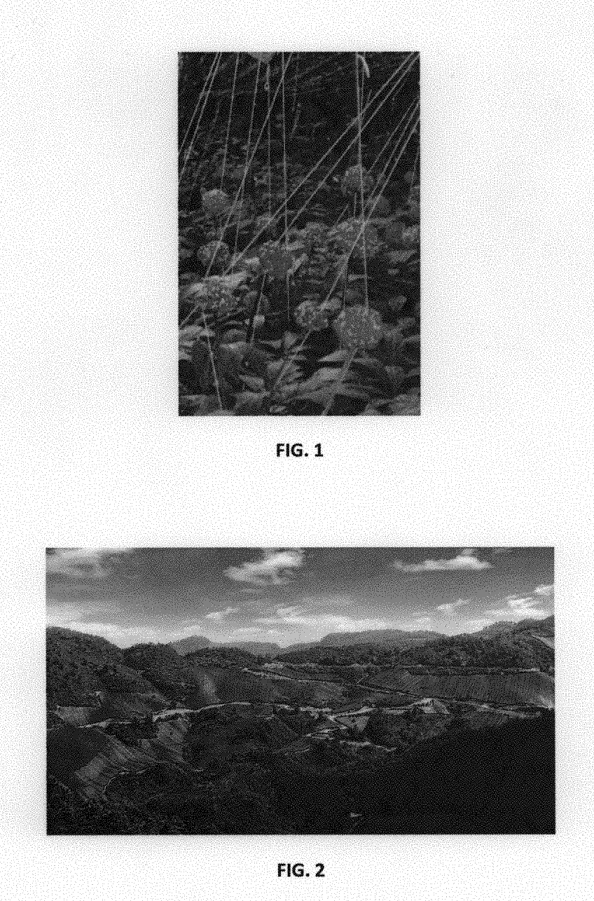

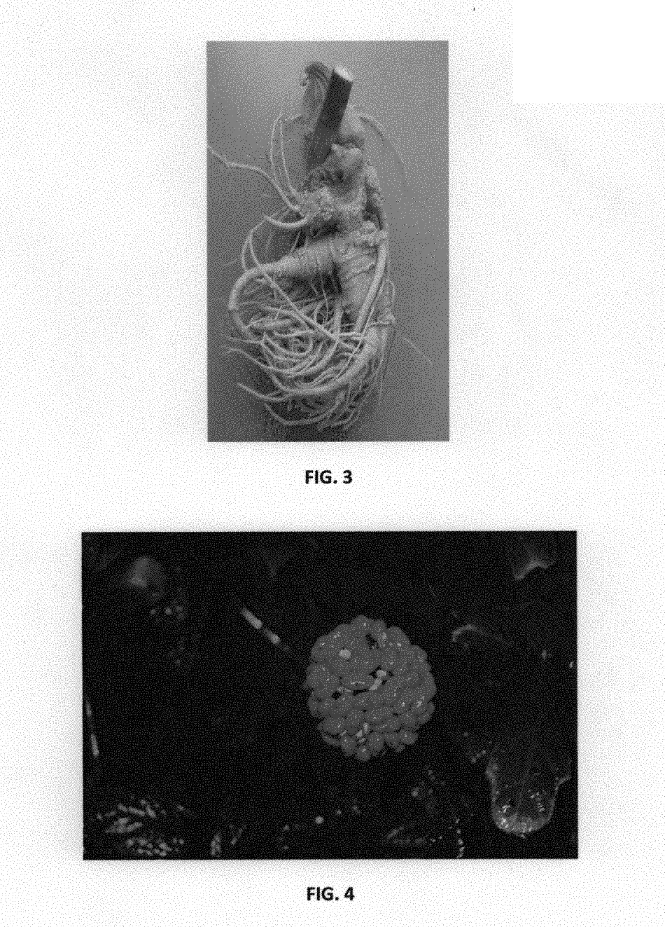

### DESCRIPTION

The invention relates to a new and distinct Wuliang Sanqi (Latin name: Panax notoginseng (Burkill) F. H. Chen) which is a cultivated geography provenance.

The Wuliang Sanqi is characterized by having a well-developed main root, fleshy hypertrophy, commonly known as “head” or “root”, one to several thick side roots on the main root, also known as “branch roots” or “ribs”, fibrous roots commonly known as “hair roots”, rhizome also known as “notch”, “lut” or “sheep intestine head”, wherein the base portion of the rhizome has thinner stem section stripes, commonly known as “parrot mouth”.

In spring of the second year, new buds sprout and the old stem of last year falls off that forms a dent on the rhizome, known as “stem marks”. Rhizome is elongated year by year, the age of the Wuliang Sanqi is known according to the number of stem marks on the rhizome.

The Wuliang Sanqi is generally has a single stem, having an enlarged stem base, commonly known as “chicken leg”, wherein the stems is generally in green color, or may also be in purple or green purple color and etc. A three-year-old Wuliang Sanqi has a plant height of 30-80 cm and a flower stalk length of 10-30 cm. The Wuliang Sanqi generally has five palmately compound leaves with each petiole having a length of 5-7 cm, and five to seven small leaves with each petiole having a length of 2 cm. The Wuliang Sanqi has leaf membranous, the central one being the largest, each having a long oval shape to an obovate-long oval shape, a length of 6.5-11 cm, a width of 2-4 cm, an acuminate to long acuminate apex, a round to wide cuneate base, leaves edge with serrates, and hairs on veins on both sides.

The Wuliang Sanqi flowers and fruits from its second year, flowering for 7 months to 9 months, wherein each of the flower stems has a length of 9-27 cm and, sometimes, a flower stem makes up about a ½ total height of the plant. Its umbel is born at the cauline top. Generally, the Wuliang Sanqi has more than one hundred of flowers, which are small, yellow-green in color, and bisexual. Each of the flowers has five calyxes, which are blade lobed and slightly triangle in shape, each having teeth at sepals margin thereof Each of the flowers has five petals, white in color, oval shaped, and arranged in compound tile form. Each of the flowers has five stamens, stylus twice to three cleft, base grow separatedly or together, ovary at lower portion, and 2-3 loculed.

The Wuliang Sanqi fruits from September to November. The fruit is drupaceous berry, in kidney or spherical kidney shape, and has a diameter of about 6-13 mm. The matured fruit is bright red, occasionally yellow or black, shiny. Seeds 1-3 tablets.

The natural lifespan of the seeds of the Wuliang Sanqi is about 15 days. The seeds need to go through a dormant period, and then accelerating germination. It takes more than 3 years from sowing to harvest, including one year of seedling (commonly known as “sub-strip”). After the sub-strip is transplanted for 2 years, harvest is available.

North Wuliang Mountain, also known as Mengle Mountain, is a branch of the southern section of the Hengduan Mountains, located in the Hengduan Mountains and the Yunnan-Guizhou Plateau, the western Yunnan Burma North Shan State Highland, the Indochina Peninsula mountain and other geographical areas of the integration. The natural conditions of Wuliang Mountain are complex, with significant north-south transition, east-west intersection characteristics, which has rich forest resources and diverse ecological environment. Nanjian Wuliang Mountain has a special geographical location, complex geological and geographical, diverse types of climate, which not only gave birth to a wealth of animal and plant resources, but also a rare blend of plants. It provides a unique condition for the Wuliang Sanqi germplasm resources.

The Wuliang Sanqi is planted at the mountainous areas having an altitude of 2000 m-2600 m. Comparing with Wenshan and other traditional Panax notoginseng planting areas, the latitude and altitude of Wuliang Mountain are higher. The forest vegetation of subtropical evergreen broad-leaved forest and semi-humid evergreen broad-leaved forest are well developed, and the ecological environment is diverse and small fragmentation environments are more. There is a high forest coverage index, abundant rainfall, cloud-covered in the dry season, high-humidity air, heavy wetting-back in night, morning fog dissipating in noon. And winter frost suppresses the growth of pests and diseases in the plantation, and spring temperature rises later, which avoids the invasion of the late spring. Warm and cool in the plantation are balanced, and the light and humidity are moderate, so the ecological environment is suitable for the growth of the Wuliang Sanqi.

The soil for Wuliang Sanqi is yellow brown soil of Wuliang Mountain, which is rich in organic matter, sometimes mixed with gravel, soft soil and highly viscous red soil. Adding humus and ripening fertilizer in the soil matrix, organic fertilizer can increase fertility of soil, is conducive to the growth of Wuliang Sanqi and root development.

The planting of the Wuliang Sanqi does not use pesticides and fertilizers, continuous planting in the same land for many years, growing well, lush growth, no serious illness and obvious continuous cropping obstacles. Its resistance to disease and resistance to continuous cropping is significantly higher than other regions. The Wuliang Sanqi has been experienced for a long-term reproductive isolation, retained the genetic characteristics, the formation of a specific geographical provenance.

The cultivation of the Wuliang Sanqi is introduced into the concept of ecological agriculture, to select the appropriate small environment, adjust measures to local conditions, and employ modern agricultural cultivation and management model, without fertilizing and pesticides, irrigation in the dry season and waterlogging in the rainy season.

According to the requirements of “Chinese Pharmacopoeia” (2010 1^{st} edition), the quality of the underground medicinal parts of 3 years, 4 years, 5 years and 7 years Wuliang Sanqi is analyzed by utilizing the HPLC quantitative analysis technique. The results demonstrate that, in 7 years Wuliang Sanqi, the content of saponin is 24.51%, wherein the content of the main root is 26.24%, the content of the big root is 22.69%, the content of the lateral root is 18.51%, the content of the fibrous root is 8.25%, and the average saponin content of the whole root of the Wuliang Sanqi is 22.40%, significantly higher than that of the 5 year old Wuliang Sanqi (17.07%), 3-year-old Wuliang Sanqi (8.01%), and 3-year-old Sanqi planted in Wenshen (8.05%) and 4-year-old Sanqi planted in Wenshen (13.75%).

A single plant of 4 years old Wuliang Sanqi has a dry weight of up to 35.05 g. The dry weight of its main root: the dry weight of the whole root is 76.46%. The saponin content of the snap portion is 18.38%, the saponin content of the main root is 13.25%, the saponin content of the lateral root was 15.05%, and the saponin content of the fibrous root was 7.05%. The ginsenoside Rg1 content of the snap portion is 10.24% and the average content is 7.29%. The highest content of Rb1 is 10.22% and the average content is 5.86%. The highest Rg1 content of the main root of a single plant is 14.46% and the average content is 6.11%. The highest content of Rb1 is 9.68% and the average content is 4.43%. The Notoginsenoside R1 content of the snap portion of a single plant is 6.93% and the average content is 3.04%. The highest Notoginsenoside R1 content of the main root of a single plant is 5.2% and the average content is 2.28%. The highest ginsenoside Re content of the snap portion of a single plant is 4.57% and the average content is 0.82%. The highest ginsenoside Re content of the main root of a single plant is 4.7% and the average content is 0.38%. The highest Rd content of the snap portion of a single plant is 1.61% and the average content was 0.89%. The highest Rd content of the main root of a single plant was 1.61% and the average content was 0.76%.

The results of the experimental analysis showed that, compared with Wenshan Sanqi herbs, the saponins composition of the Wuliang Sanqi was the same as the Wenshan Sanqi. The saponin contents of the snap portion, the main root, the big root, and the lateral root of the 3 years old Wuliang Sanqi is in line with the requirements of the National Pharmacopoeia. The saponin content of the 4 years old Wuliang Sanqi is significantly higher than that of the 3 years old Wuliang Sanqiu and the Wenshan Sanqi (3 years old). The Wuliang Sanqi is a high-quality herb. In addition, the yield of the Wuliang Sanqi is 150-200 kg/mu in average.

A planting method of the Wuliang Sanqi includes the following steps:

1. Selection of properly forest vegetation: Select a land parcel, wherein tall trees, shrubs and grass live the surface thereof. Wuliang Sanqi grows with grass, and a forest can provides a larger transpiration and protection than an open space, which defines a natural greenhouse to a relatively stable airflow, temperature and a relatively stable change of humidity. The forest may be mainly subtropical humid evergreen broad-leaved forest and semi-humid evergreen broad-leaved forest, and meantime includes warm coniferous forest, moss and dwarf forest and other vegetation types of the original forest vegetation, for example, Taxus chinensis, taxus chinensis var mairei, Bretschneidera sinensis, Alcimandra cathcardii, Cyathea chinensis, Brainea insignis, Tetracentron sinense, Torreya yunnanensis, Caulis Spatholobi, Cerasus cerasoides and Western Yunnan purple tree (Latin name: Nyssa shweliensis (W. W. Smith) Airy-Shaw).

2. Selection of terrain: The land parcel may have an elevation of 2000˜2600 m or more, a slope of 30°˜60°, the slope of 50° is most preferably for Wuliang Sanqi, and the Wuliang Sanqi is preferably planted in the hillside thereof A hummock and a foot of a hill should not be selected.

An eastern slope, southeast slope, northern slope or northeast slope can be chosen. Preferably, a ditch, a partial shade or full shade area of a northern slope or east slope of a river are chosen. Most preferably, a northeast slope can be chosen. Strive to choose a land parcel having a high flatness, and preferably, being gently.

3. Choosing forest soil: Choose forest yellow soil and yellow brown soil, which are rich in organic matter, slightly lower in salt saturation, and have a total porosity greater than 50%, good permeability and good exchange performance of soil.

4. Selection of cold-resistance population: Breed the Wuliang Sanqi seeds, which have been grown in a relatively enclosed mountain environment for a long time, and that, since the population grows in the mountains and valleys for a long time, it has strong competition, cold resistance and disease resistance capabilities in bad natural environment.

5. Clearing forest land: Cut off the shrub weeds in the forest land before seeding, wherein dry leaves and grass are properly preserved for preventing drought and moisturizing, wherein the canopy intensity of light is 0.7 to 0.8, and that, as a shady plant, dense trunk is destroy to ensure that 20%˜30% of the light transmittance.

6. Making a hole and seeding naturally therein: The first seeding employs artificial hole sowing which takes no damage to the arable layer, and the seeding time may be in December to January of the following year and the seeding is done by line spacing 4×5 cm and 2-3 cm deep hole in the forest, wherein every 1˜2 seeds are seeded in a hole. Hereinafter, breeding mainly relies on seeds mature landing, natural germination into seedlings and natural reproduction thereof.

7. Management of lightly and minimizing manual intervention: Weeds which seriously impact the growth of the Wuliang Sanqi can be cut with scissors grass, leaving 5-8 cm bottom, to not destroy the original ecological environment as a prerequisite. Generally, when the wild Wuliang Sanqi sprouts, no weeding, no fertilizing, no pesticide spraying, no herding is provided and people cannot walk around in the forest. And trees don't be cut to prevent the growth of wild Sanqi being influenced.

8. Prevention of diseases and insects by utilizing plant-derived pesticides: The main disease of SANQI herb is powdery mildew, the main pests are aphids, which mostly appear in rainy and high temperature seasons, and the diseased plants should be immediately removed diseased plants, and accumulating and fermenting pecan leaves and camphor leaves (1:1 ratio) ratio, and then sprinkling the fermented mixture around SANQI herbs. Or preparing decoction solution by utilizing Sophora flavescens, Radix Stemonae and Melia azedarach leaves (2:1.5:1.5 ratio), selecting to spray pesticides on the leaf surfaces by a sprayer on a clear day, so as to sterilize and prevent diseases and deinsectization.

9. Covering with grass to improve soil fertility and moisturize: Weeding and topping is not provided for a long-term during the growing period of SANQI herb to prevent SANQI herb being damaged. Weeding may break lateral roots of SANQI. After weeding and topping, harmful bacteria may enter into the pant from the damaged position. Covering by utilizing weeds and pecan leaves to increase an organic matter content in the soil, reduce soil moisture evaporation, and keep the soil moisture, which is conducive to the growth of wild Sanqi.

10. Managing to overwinter safely: Before the weather becomes cold, overwhelming withered stems and covering firewood and leaves on the withered stems, and then covering some soil thereon to prevent buds being frostbite and overwinter safely, so that SANQI can early sprout, early sprout and obtain strong buds.

11. Harvesting when being matured: The longer the wild Sanqi herbs accumulate nutrients, the better its quality is, and the growth period is at least 10 years, before they can be digged. When the leaves of Wuliang Sanqi herbs become from green to yellow in late October to early November, remove stems and leaves, fibrous roots and soil, digging and drying up.

What is claimed is:

1. A new and distinct Sanqi plant known as ‘Wuhang Sanqi’ having the characteristics substantially as illustrated and described herein.
